(19) United States
(12) Patent Application Publication
(10) Pub. No.: US 2026/0294788 A1
(43) Pub. Date: Oct. 1, 2026

(54) COSMETIC TREATMENT METHOD WITH LED

(71) Applicant: Conopco, Inc., d/b/a UNILEVER, Englewood Cliffs, NJ (US)

(72) Inventors: Mariangela GOMES DE OLIVEIRA SICHMANN, Campinas (BR); Angela Mary MURPHY, Los Angeles, CA (US); John Chun Sing NIP, Shelton, CT (US); Xuyen PHAM, Torrance, CA (US); Sheila Alves ROCHA-GUSHIKEM, Bogota, NJ (US); Stella VILLA-MORA, Oakland, NJ (US)

(73) Assignee: Conopco, Inc., d/b/a UNILEVER, Englewood Cliffs, NJ (US)

(21) Appl. No.: 18/998,207

(22) PCT Filed: Jul. 18, 2023
(86) PCT No.: PCT/EP2023/069922

§ 371 Date: Jan. 24, 2025

Related U.S. Application Data
(60) Provisional application No. 63/393,956, filed on Jul. 31, 2022.

(30) Foreign Application Priority Data
Sep. 20, 2022 (EP) 22196710.2

(51) Int. Cl. A61K 8/9789; A61K 8/9711; A61Q 19/08

(52) U.S. Cl. CPC A61K 8/9789; A61K 8/9711; A61Q 19/08; A61K 2800/81; A61K 2800/87

(57) ABSTRACT

The present invention relates to a method for the cosmetic treatment of human skin, in particular for the stimulation of procollagen production. The present invention also relates to cosmetic treatment kits for executing the disclosed method.

### FIELD OF THE INVENTION

The present invention relates to a method for the cosmetic treatment of human skin, in particular for the stimulation of procollagen production. The present invention also relates to cosmetic treatment kits for executing the disclosed method.

### BACKGROUND OF THE INVENTION

Collagen is an important scaffold protein that contributes to a youthful appearance by maintaining smoothness and elasticity of skin. Dermal fibroblasts in skin synthesize a protein precursor to collagen known as procollagen that is subsequently secreted by the cell and assembled into collagen fibrils. It is highly desirable to enhance the production of collagen in skin. Unfortunately, the inherent ability to produce collagen declines with age, leading to an undesirable loss in firmness and elasticity of skin. However, conventional means like topically applying cosmetic compositions to the skin are typically inferior because the collagen molecule has a molecular weight that is too large to penetrate the top layer of skin (i.e., the epidermis) and is thus not sufficiently absorbed. Therefore, the present inventors have identified a need for a cosmetic treatment method for stimulating procollagen production, and hence collagen, within human skin.

### SUMMARY OF THE INVENTION

Accordingly, in a first aspect, there is provided a non-therapeutic method for increasing procollagen production of skin comprising the steps of:
(a) topically applying to a surface of skin in need of treatment a cosmetic composition comprising from (i) 0.001 to 10%, or 0.005 to 8%, or 0.01 to 6% by weight of seaweed-derived active, from (ii) 0.001 to 10%, or 0.005 to 8%, or 0.01 to 6% by weight of a first botanical active, from (iii) 0.001 to 10%, or 0.005 to 5%, or 0.01 to 3% by weight of a second botanical active, and (iv) a cosmetically acceptable vehicle; and
(b) applying to the skin a source of light radiation originating from a light-emitting diode (LED) having wavelength ranging from 300 nm to 800 nm, or 400 nm to 700 nm, or 500 nm to 680 nm, or 600 nm to 660 nm, for less than 2 hours, or 10 to 60 minutes, or 20 to 45 minutes, or 20 to 40 minutes, or 25 to 35 minutes.

In a second aspect of the disclosed method for increasing procollagen, there is provided an optional step (c) wherein excess of the cosmetic composition topically applied to the skin after exposure to light radiation may be removed, or wiped or rinsed off.

In a third aspect, there is provided a composition comprising:
(a) 0.001 to 10%, or 0.005 to 8%, or 0.01 to 6% by weight of seaweed-derived active,
(b) 0.001 to 10%, or 0.005 to 8%, or 0.01 to 6% by weight of a first botanical active,
(c) 0.001 to 10%, or 0.005 to 5%, or 0.01 to 3% by weight of a second botanical active, and
(d) a cosmetically acceptable vehicle.

In a fourth aspect, there is provided use of a composition comprising a seaweed-derived active, a first botanical active, and a second botanical active for increasing procollagen-I production of the skin.

In a fifth aspect, there is provided a cosmetic treatment kit for executing the disclosed method of the first and second aspects, wherein the kit comprises a cosmetic composition comprising (i) 0.001 to 10%, or 0.005 to 8%, or 0.01 to 6% by weight of seaweed-derived active, (ii) from 0.001 to 10%, or 0.005 to 8%, or 0.01 to 6% by weight of a first botanical active, (iii) from 0.001 to 10%, or 0.005 to 5%, or 0.01 to 3% by weight of a second botanical active, (iv) a cosmetically acceptable vehicle, and (v) a device for cosmetic treatment by light comprising at least one source of light-emitting diode (LED) light radiation at a wavelength of 300 nm to 800 nm, or 400 nm to 700 nm, or 500 nm to 680 nm, or 600 nm to 660 nm.

All other aspects of the present method, cosmetic composition, and cosmetic treatment kit will become more readily apparent upon considering the detailed description and examples which follow.

### DETAILED DESCRIPTION OF THE INVENTION

Unless explicitly stated otherwise, all ranges described herein are meant to include all ranges subsumed therein. Except in the operating and comparative examples, or where otherwise explicitly indicated, all numbers used in this description indicating amounts, or ratios of materials and/or use thereof are to be understood as modified by the word “about.”

For the avoidance of doubt, the term “comprising” is meant not to be limiting to any stated elements but rather to encompass non-specified elements of major or minor functional importance. Therefore, the listed steps, elements or options need not be exhaustive. Whenever the words “including” or “having” are used, these terms are meant to be equivalent to “comprising” as defined above. The term comprises is also meant to encompass the terms consisting essentially of and consisting of. For the avoidance of doubt, a composition comprising seaweed-derived active, a first botanical active, a second botanical active, and a cosmetically acceptable vehicle is meant to include a composition consisting essentially of the same and a composition consisting of the same. As to the percentages used herein, the same are meant to be by weight of ingredient active, unless noted otherwise.

The disclosure, as found herein, is to be considered to cover all embodiments as found in the claims as being multiply dependent upon each other irrespective of the fact that claims may be found without multiple dependency or redundancy.

In a first aspect, there is provided a non-therapeutic method for increasing procollagen comprising the steps of:
(a) topically applying to a surface of skin in need of treatment a cosmetic composition comprising from (i) 0.001 to 10%, or 0.005 to 8%, or 0.01 to 6% by weight of seaweed-derived active, (ii) from 0.001 to 10%, or 0.005 to 8%, or 0.01 to 6% by weight of a first botanical active, (iii) from 0.001 to 10%, or 0.005 to 5%, or 0.01 to 3% by weight of a second botanical active, and (iv) a cosmetically acceptable vehicle; and
(b) applying to the skin a source of light radiation originating from a light-emitting diode (LED) having wavelength ranging from 300 nm to 800 nm, or 400 nm to 700 nm, or 500 nm to 680 nm, or 600 nm to 660 nm, for less than 2 hours, or 10 to 60 minutes, or 20 to 45 minutes, or 20 to 40 minutes, or 25 to 35 minutes.

Non-therapeutic, as used herein, includes enhancing the procollagen production in skin to yield a younger and smoother looking skin.

In one aspect of the disclosed method for increasing procollagen, there is provided an optional step (c) wherein excess of the cosmetic composition topically applied to the skin after exposure to light radiation may be removed, or wiped or rinsed off.

In another aspect, the disclosed method of the first aspect comprises step (a) followed by step (b), optionally followed by step (c). In still another aspect, the disclosed method of the first aspect comprises step (b) followed by step (a), optionally followed by step (c).

The cosmetic composition, as disclosed herein, comprises (i) an active derived from seaweed, (ii) a first botanical active, and (iii) a second botanical active. The term “active” or “active ingredient,” as used herein, refers to an ingredient included in a composition specifically to improve a desired skin condition, such as photoaging, by providing a benefit to the skin.

Seaweed, which generally belong to the phylum Ochrophyta, encompasses numerous species of marine algae. Actives derived from seaweed, particularly those of the order Laminariales, are suitable for use and may be found in extract form. The seaweed-derived active includes those from Laminaria cloustoni, Laminaria digitata, Laminaria hyperborea, Laminaria japonica, Laminaria ochroleuca, Laminaria saccharina, and mixtures thereof. In one embodiment, the seaweed-derived active comprises Laminaria digitata extract. The cosmetic composition, as described herein, comprises 0.001 to 10%, or 0.005 to 8%, or 0.01 to 6%, or 0.01 to 5%, or 0.02 to 4%, or 0.03 to 3%, or 0.04 to 2.5%, or 0.1 to 2.5%, or 0.5 to 2.5%, or 1 to 2.25% of the seaweed-derived active by weight of the cosmetic composition.

In an embodiment, the seaweed-derived active is further supplemented with magnesium aspartate, disodium adenosine triphosphate, or a combination thereof. In another embodiment, the seaweed-derived active is substantially free of magnesium aspartate, disodium adenosine triphosphate, or combination thereof. “Substantially free of” as used herein, is intended to mean comprising less than 10% by weight, or less than 5% by weight, or less than 2% by weight, or less than 0.5% by weight, or less than 0.1% by weight, or 0% by weight of the active ingredient. In yet another embodiment, the seaweed-derived active is made commercially available by Chemyunion under the tradename Energilium 3R.

The first botanical active comprises a Lindera extract. In an embodiment of the first botanical active, the active comprises extracts of Lindera aggregate, Lindera latifolia, Lindera obtusiloba, Lindera strychnifolia, or mixtures thereof. In another embodiment, the active comprises Lindera strychnifolia extract, or Lindera strychnifolia (root) extract. In still another embodiment, the active is Lindera strychnifolia (root) extract. The first botanical active may optionally be further supplemented with magnesium aspartate, disodium adenosine triphosphate, or a combination thereof. In another aspect, the first botanical active is substantially free of magnesium aspartate, disodium adenosine triphosphate, or combination thereof. In yet another aspect, the first botanical active is Lindera strychnifolia (root) extract made commercially available by SILAB under the tradename Circagenyl®. The cosmetic composition, as described herein, comprises 0.001 to 10%, or 0.005 to 8%, or 0.01 to 6%, or 0.02 to 5%, or 0.03 to 4%, or 0.04 to 3.5%, or 0.1 to 4%, or 1 to 3.5%, or 2 to 3.25% of the first botanical active by weight of the cosmetic composition.

The second botanical active comprises an olive extract. The olive extract may be Olea europaea (olive) leaf extract, Olea europaea fruit oil (olive oil), or a combination thereof. In an embodiment, the second botanical active may optionally be further supplemented with magnesium aspartate, disodium adenosine triphosphate, or a combination thereof. In another embodiment, the second botanical active is substantially free of magnesium aspartate, disodium adenosine triphosphate, or combination thereof. In yet another embodiment, the first botanical active is Olea europaea (olive leaf) extract made commercially available by Barnet Products LLC under the tradename Olivys-EA. The cosmetic composition, as described herein, comprises 0.001 to 10%, or 0.005 to 5%, or 0.01 to 3%, or 0.02 to 2.5%, or 0.03 to 2%, or 0.04 to 1.5%, or 0.1 to 2%, or 0.5 to 1.5%, or 0.1 to 1%, or 1 to 1.4% of the second botanical active by weight of the cosmetic composition.

The disclosed cosmetic compositions herein comprise a weight ratio of seaweed-derived active to first botanical active ranging from 1:3 to 4:5, or 1:2 to 3:4, or 3:5 to 7:10.

The disclosed cosmetic compositions herein comprise a weight ratio of seaweed-derived active to second botanical active ranging from 5:2 to 1:1, or 2:1 to 5:4, or 3:2 to 9:5.

Cosmetic compositions disclosed herein may also comprise a cosmetically acceptable vehicle to act as a moisturizer, diluent, dispersant, or carrier for the skin benefit agents in the composition, so as to facilitate its distribution when the composition is applied to the skin. This cosmetically acceptable vehicle may be aqueous, anhydrous (and suitable for dilution), or an emulsion. Oily carriers (i.e., nonpolar) in the presence of water and an emulsifier will form emulsion systems as carriers. In one aspect, the compositions are aqueous or an emulsion, (i.e., water-in-oil or oil-in-water or a double emulsion). The cosmetic compositions ordinarily will be, for example, a cream, serum, spray, balm, deodorant, or lotion. In one aspect, the composition is a cream or lotion.

Desirable carriers employed include water, sea water, rosewater (e.g., Rosa damascena flower Water), tea (e.g., Camellia sinensis Leaf Water), Aloe Barbadensis (aloe vera) leaf juice, witch hazel (e.g., Hamamelis virginiana extract), lavender water (e.g., Lavendula angustifolia flower water), grape water (e.g., Vitis vinifera fruit water and Vitis vinifera juice). In one aspect, water is the carrier. Amounts of water may range from 5 to 85%, or from 15 to 75%, or from 30 to 70%, based on the total weight of the total cosmetic composition. The cosmetically acceptable vehicle can, in the absence of other cosmetic adjuncts, form the balance of the composition. Besides water, suitable carrier classes include, but are not limited to, silicones, polyhydric alcohols, fatty acids, hydrocarbons, triglycerides, waxes, and thickening agents.

It is within the scope of the disclosed cosmetic composition to contain silicones, which may be categorized into the volatile and nonvolatile variety. Amounts may range, for example, from 0.01 to 25%, or 0.1 to 20% by weight of the cosmetic composition. In an embodiment of the cosmetic composition, 0.01 to 2% by weight of the silicone is contained in the composition with no significant impact on sensory properties. The term “volatile” as used herein refers to those materials which have a measurable vapor pressure at ambient temperature. Volatile silicone oils for optional use are chosen from cyclic (cyclomethicone) or linear polydimethylsiloxanes containing from 3 to 9, or from 4 to 5, silicon atoms.

Nonvolatile silicones useful in this composition include polyalkyl siloxanes, polyalkylaryl siloxanes, and polyether siloxane copolymers. The essentially nonvolatile polyalkyl siloxanes useful herein include, for example, polydimethyl siloxanes with viscosities from about 5×10⁻⁶ to 0.1 m²/s at 25° C. Emulsifying and non-emulsifying silicone elastomers are also suitable for use in the cosmetic composition disclosed. In one embodiment, the composition may be substantially free of silicones.

Conventional humectants, generally of the polyhydric alcohol-type materials, may be used in the disclosed cosmetic composition. Typical polyhydric alcohols include glycerol (i.e., glycerine or glycerin), propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. In one embodiment, the cosmetic composition comprises glycerin, propylene glycol or a mixture thereof. The amount of humectant employed may range anywhere from 0.1 to 20%, or 0.5 and 15%, or between 1 and 7% by weight of the cosmetic composition.

Waxes and wax esters are also desirable for use. These waxes include animal-derived waxes or those from animal byproducts (e.g., beeswax, spermaceti wax, Chinese wax, wool wax, shellac wax), plant-derived waxes (e.g., tribehenin wax, carnauba wax, candelilla wax, bayberry wax, jojoba wax, orange wax, rice bran wax, sunflowerwax, castorwax, soy wax), mineral waxes (e.g., montan wax, ceresin wax, ozokerite) and synthetic derivatives of natural waxes. The amount of wax and wax esters employed may range from 0.01 to 10%, or from 0.1 to 8% by weight of the composition.

Hydrocarbons may be optionally included in the cosmetic composition disclosed herein. Desirable hydrocarbons may include mineral oil, petrolatum, and polyalpha-olefins. Examples of preferred volatile hydrocarbons include polydecanes such as isodecane and isododecane and the C₇-C₈ through C₁₂-C₁₅ isoparaffins.

Cosmetic compositions disclosed herein may optionally comprise one or more thickening agents at inclusion levels from 0.05 to 10%, or from 0.1 to 5%, or from 0.25 to 4%, by weight of the composition. Useful thickeners include polysaccharides, which comprise of starches, natural/synthetic gums and cellulosics. Desirable starches include tapioca starch, cornstarch, potato starch, aluminum starch octenylsuccinate, and sodium hydroxypropyl starch phosphate. Desirable gums include xanthan, sclerotium, pectin, karaya, arabic, agar, guar, carrageenan, alginate, and combinations thereof. Desirable cellulosics include hydroxypropyl cellulose, hydroxypropyl methylcellulose, ethylcellulose, and sodium carboxy methylcellulose. Synthetic polymers functioning as thickening agents are also desirable for use, including polyacrylamides, Carbomers, and taurate copolymers. Citric fibers may also be used.

Emulsifiers may be present in the disclosed cosmetic composition. Emulsifiers desirable for use will have an HLB from 2.5 to 17. The HLB of the emulsifier used when oil-continuous emulsions are desired will be 2.5 to 7.5, or from 3 to 6.5, or from 3 to 6. The HLB of the emulsifier used when water-continuous emulsions are desired will be 8 to 17, or from 8.5 to 15, or from 9 to 14.

Total concentration of the emulsifier may range from 0.1 to 20%, or from 1 to 10%, or from 1 to 8% by weight of the composition. The emulsifier may be selected from the group consisting of anionic, nonionic, cationic and amphoteric surfactants. Desirable nonionic surfactants are those with a C₁₀-C₂₀ fatty alcohol or acid hydrophobe condensed with from about 2 to about 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also desirable nonionic emulsifiers. Still other nonionic surfactants include glyceryl stearate, glycol stearate and stearamide AMP.

Desirable anionic emulsifiers include alkyl ether sulfate and sulfonates, alkyl sulfates and sulfonates, alkylbenzene sulfonates, alkyl and dialkyl sulfosuccinates, C₈-C₂₀ acyl isethionates, C₈-C₂₀ acyl methyl isethionates, C₈-C₂₀ acyl methyl taurates, C₈-C₂₀ alkyl ether phosphates, alkyl ether carboxylates, and combinations thereof.

Cationic emulsifiers that may be used include, for example, palmitamidopropyltrimonium chloride, distearyldimonium chloride, diester quaternary ammonium compounds (e.g. distearoylethyl dimonium chloride), and mixtures thereof.

Useful amphoteric emulsifiers can include cocoamidopropyl betaine, C₁₂-C₂₀ trialkyl betaines, sodium lauroamphoacetate, and sodium laurodiamphoacetate or a mixture thereof.

Fragrances, fixatives, abrasives and additional skin benefit agents may optionally be included in cosmetic compositions of the present invention. Skin benefit agents suitable for use in this invention are meant to include but not be limited to opacifiers, colorants, humectants, emollients, occlusive agents, plant extracts, optical agents, skin lightening agents, anti-inflammatory agents, anti-acne agents, antioxidants, sunscreens, photostabilizers, surfactants, wrinkle reducing agents, coloring agents, desquamation promoters, exfoliating agents, mixtures thereof or the like. Each of these substances may range from 0.05 to 5%, or between 0.1 and 3% by weight.

Other additional optional skin benefit agents desirable for use in this invention include minerals and skin nutrients such as milk; magnesium, calcium, copper, zinc and other metallic components; alpha hydroxy acids, beta hydroxy acids, e.g., salicylic acid and derivatives thereof (such as 5-octanoyl salicylic acid, heptyloxy 4 salicylic acid, and 4-methoxy salicylic acid); kojic acid; hydroquinone and arbutin; resveratrol; saccharide isomerate; undecylenoyl phenylalanine; resorcinol derivatives (including thiamidol, 4-ethyl resorcinol, 4-isopropyl resorcinol, 4-hexyl resorcinol, 4-cyclopentyl resorcinol, 4-cyclohexyl resorcinol and acylated forms thereof); ceramides (e.g., Ceramide 1, Ceramide 3, Ceramide 3B and Ceramide 6) and pseudoceramides; allantoin; pyroglutamic acid (PCA) salt derivatives including zinc PCA and sodium PCA; and skin benefit acids and derivatives thereof (12-hydroxystearic acid; petroselinic acid; conjugated linoleic acid; octadecanoic acid; hyaluronic acid and its salt derivatives); retinol and derivatives thereof (retinyl proprionate, retinyl palmitate, retinyl acetate), vitamins and derivatives thereof (Vitamin B₂, Vitamin B₃ (niacinamide), picolinamide, Vitamin B₅ (panthenol), Vitamin B₆, Vitamin C, ascorbyl phosphate, ascorbyl glycoside, Vitamin D, Vitamin E, tocopheryl acetate, tocopheryl palmitate, tocopheryl lincoleate, folic acid, Vitamin K, and biotin), mixtures thereof and the like. Such skin benefit agents, when used, collectively make up from 0.001 to 12%, or 0.05 to 7% by weight of the cosmetic composition. Antibacterial agents like terpineol, thymol, and low molecular weight acids may also be optionally included. Other agents suitable for optional use include zinc pyrithione, octopirox, or mixtures thereof.

A wide selection of botanical extracts may optionally be included in cosmetic compositions disclosed herein. The extracts may either be soluble in water or oil, carried in a solvent that is hydrophilic or hydrophobic, respectively. In one embodiment, water or ethanol are the extract solvents. Illustrative examples include those extracted from green tea, yarrow, chamomile, licorice, aloe vera, citrus unshui, willow bark, alfalfa, algae, grape seed, witch hazel, sage, thyme and rosemary, as well as oils such as those derived from sea buckthorn, moringa, argan, avocado, calendula, algal, and marula. Soy extracts may be used and especially when it is desirable to include retinol. Such extracts, when used, are employed individually or collectively in amounts ranging from 0.001 to 12%, or 0.01 to 7%, or 0.02 to 5%, or 0.03 to 4% by weight of the cosmetic composition.

It is within the scope of the disclosed cosmetic composition to optionally include sunscreens and photostabilizers. The sunscreens and photostabilizers that may be used include such materials as octylmethoxycinnamate (OMK), ethylhexyl salicylate, phenylbenzimidazole sulfonic acid (Ensulizole), ethylhexyl p-methoxycinnamate, available as Parsol MCX®, Avobenzene (butyl methoxydibenzoylmethane), available as Parsol 1789© and benzophenone-3, also known as oxybenzone. Inorganic sunscreen actives may be employed such as microfine titanium dioxide, zinc oxide, polyethylene, and various other polymers are also desirable. Other sunscreens desirable for use include p-aminobenzoic acid (PABA), octyldimethyl-PABA, 2-ethoxyethyl p-methoxy cinnamate, benzophenone-1, benzophenone-2, benzophenone-6, benzophenone-8, benzophenone-9, benzophenone-12, homomethyl salicylate, menthyl anthranilate, benzophenone-4, triethanolamine salicylate, terephthalylidene dicamphor sulfonic acid, bisoctriazole, bisethylhexyloxyphenol methoxyphenyl triazine, bisdisulizole disodium, diometriazole trisiloxane, octyltriazone, iscotrizinol, polysilicone-15, isopentenyl-4-methoxycinnamate, mixtures thereof or the like. Also suitable for use is octocrylene. Amounts of the sunscreen or photostabilizing agents when present may generally range from 0.1 to 30%, or from 0.5 to 20%, or from 0.75 to 10% by weight of the cosmetic composition.

Cosmetic compositions disclosed herein may also include natural and synthetic antioxidants. Illustrative but nonlimiting examples of antioxidants for use in the composition include butylated hydroxytoluene (BHT), dilauryl thiodipropionate, pentaerythrityl tetra-di-t-butyl hydroxyhydrocinnamate, and polyphenols. The total weight percent of antioxidants comprised is from 0.01 to 4%, or from 0.02 to 3%, or from 0.05 to 2%.

Another optional additive suitable for use includes hemp oil with 2.5 to 25% by weight cannabigerol and/or cannabidiol at from 0.5 to 10 percent by weight. When used, such oil makes up from 0.0001 to 12%, or from 0.01 to 5% by weight of the cosmetic composition.

Optionally, one or more coloring agent may also be included in the disclosed cosmetic compositions. Coloring agents include dyes or pigments of natural or synthetic origin. A dye colorant selected for use may be organic or inorganic and water-soluble or oil-soluble. Cosmetic compositions comprise from 0.01 to 20%, or 0.1 to 15%, or 0.5 to 10% by weight of the coloring agent, when used, relative to the total weight of the cosmetic composition.

Traditional buffers or pH modifiers are also suitable for inclusion to the disclosed cosmetic composition. These include common additives such as sodium hydroxide, potassium hydroxide, hydrochloric acid, citric acid, triethanolamine and aminomethyl propanol. In an embodiment, the pH of the cosmetic composition is from 4 to 8, or from 4.25 to 7.75, or 5 to 7.5. Viscosity of the cosmetic composition is from about 1,000 to about 200,000 cps, or from about 5,000 to about 180,000 cps, or from about 10,000 to about 150,000 cps, taken under conditions of 25° C. and a shear rate of 1 s⁻¹ with a strain controlled parallel plate rheometer made commercially available from suppliers like T.A. Instruments under the Discovery name. Alternatively, viscosity can also be measured using a Brookfield Viscometer (speed at 20 rpm, spindle 5, helipath off, for one (1) minute at 25° C.). The cosmetic composition can be formulated as a lotion having a viscosity from 4,000 to 10,000 mPas, a fluid cream having a viscosity from 10,000 to 20,000 mPas or a cream having a viscosity from 20,000 to 200,000 mPas or above.

Preservatives can be incorporated into the disclosed cosmetic compositions as desired to protect against the growth of potentially harmful microorganisms. Cosmetic chemists are familiar with appropriate preservatives and routinely choose them to satisfy preservative tests and product stability tests. Preservative systems should be selected having regard for the use of the composition and possible incompatibilities between the preservatives and other ingredients in the formulation. Exemplary examples of preservatives for compositions of this invention include, without limitations, iodopropynyl butyl carbamate (IPBC), phenoxyethanol, ethylhexylglycerine, 1,2-octanediol, methyl paraben, propyl paraben, imidazolidinyl urea, sodium dehydroacetate, propanediol, alkyl esters of para-hydroxybenzoic acid, sodium benzoate, benzoic acid, hydroxyacetophenone, DMDM hydantoin derivatives, propionate salts, and a variety of quaternary ammonium compounds. Preservatives are employed in amounts ranging from 0.01 to 2% by weight of the composition, including all ranges subsumed therein. In another aspect, no traditional preservative is used in the composition since such a cosmetic composition can be made and used on demand (from anhydrous concentrate) if desired giving the consumer the option to have a preservative-free product. In one aspect, the cosmetic composition may be free of preservative. In another aspect, the cosmetic composition may comprise of a preservative system that is formaldehyde-free, paraben-free, or both.

In use, a small quantity of the composition, for example from 1 to 100 ml, is applied to exposed areas of the skin in need of treatment, from a suitable container or applicator and, if necessary, it is then spread over and/or rubbed into the skin using the hand or fingers or a suitable device or implement.

The light radiation to which the skin in need of treatment is exposed may originate from a single light source emitting at different wavelengths or a multitude of light sources. It is desirable that the light radiation originates from a light-emitting diode (LED) having wavelength ranging from 300 nm to 800 nm, or 400 nm to 750 nm, or 500 nm to 700 nm, or 550 to 690 nm, or 600 nm to 670 nm. In a particular embodiment, the LED light radiation is red light. The light radiation may originate from an LED, from a LED matrix, from an OLED, or a combination thereof. It is further desirable that the skin in need to treatment is exposed to the LED light radiation for less than 2 hours, or about 10 to 60 minutes, or about 20 to 50 minutes, or about 20 to 40 minutes, or about 25 to 35 minutes per treatment. In one embodiment, individual sessions of LED light radiation treatments to the same surface of skin in need of treatment may be consecutive on the same day, or on subsequent days, or based on any recommended treatment schedule as recommended by professionals administering the LED treatments.

In another aspect, there is also provided a cosmetic treatment kit for executing the disclosed method (or regimen) of the first and second aspects, wherein the kit comprises a cosmetic composition comprising (i) 0.001 to 10%, or 0.005 to 8%, or 0.01 to 6% by weight of seaweed-derived active, from (ii) 0.001 to 10%, or 0.005 to 8%, or 0.01 to 6% by weight of a first botanical active, from (iii) 0.001 to 10%, or 0.005 to 5%, or 0.01 to 3% by weight of a second botanical active, (iv) a cosmetically acceptable vehicle, and (v) a device for cosmetic treatment by light comprising at least one source of light-emitting diode (LED) light radiation at a wavelength of 300 nm to 800 nm, or 400 nm to 700 nm, or 500 nm to 680 nm, or 600 nm to 660 nm.

In one embodiment, there is provided a non-therapeutic method for increasing procollagen comprising the steps of:
(a) topically applying to a surface of skin in need of treatment a cosmetic composition comprising from (i) 0.001 to 10%, or 0.005 to 8%, or 0.01 to 6% by weight of seaweed-derived active, from (ii) 0.001 to 10%, or 0.005 to 8%, or 0.01 to 6% by weight of a first botanical active, from (iii) 0.001 to 10%, or 0.005 to 5%, or 0.01 to 3% by weight of a second botanical active, and (iv) a cosmetically acceptable vehicle;
(b) applying to the skin a source of light radiation originating from a light-emitting diode (LED) having wavelength ranging from 300 nm to 800 nm, or 400 nm to 700 nm, or 500 nm to 680 nm, or 600 nm to 660 nm, for less than 2 hours, or 10 to 60 minutes, or 20 to 45 minutes, or 20 to 40 minutes, or 25 to 35 minutes; and
(c) optionally removing, or wiping or rinsing off, excess of the cosmetic composition topically applied to the skin after exposure to light radiation.

In a second embodiment, there is provided a non-therapeutic method for increasing procollagen comprising the steps of:
(a) applying to the skin a source of light radiation originating from a light-emitting diode (LED) having wavelength ranging from 300 nm to 800 nm, or 400 nm to 700 nm, or 500 nm to 680 nm, or 600 nm to 660 nm, for less than 2 hours, or 10 to 60 minutes, or 20 to 45 minutes, or 20 to 40 minutes, or 25 to 35 minutes;
(b) topically applying to a surface of skin in need of treatment a cosmetic composition comprising (i) 0.001 to 10%, or 0.005 to 8%, or 0.01 to 6% by weight of seaweed-derived active, from (ii) 0.001 to 10%, or 0.005 to 8%, or 0.01 to 6% by weight of a first botanical active, from (iii) 0.001 to 10%, or 0.005 to 5%, or 0.01 to 3% by weight of a second botanical active, and (iv) a cosmetically acceptable vehicle; and
(c) optionally removing, or wiping or rinsing off, excess of the cosmetic composition topically applied to the skin after exposure to light radiation.

In still another embodiment, there is provided a cosmetic composition comprising:
(a) 0.001 to 10%, or 0.005 to 8%, or 0.01 to 6% by weight of seaweed-derived active,
(b) 0.001 to 10%, or 0.005 to 8%, or 0.01 to 6% by weight of a first botanical active,
(c) 0.001 to 10%, or 0.005 to 5%, or 0.01 to 3% by weight of a second botanical active, and
(d) a cosmetically acceptable vehicle.

In yet another embodiment, there is provided use of a cosmetic composition comprising (i) a seaweed-derived active, (ii) a first botanical active, (iii) a second botanical active, and (iv) a cosmetically acceptable vehicle for increasing procollagen-I production of the skin.

The term “derivative(s)” as used with reference to ingredients herein refers to compounds which may be formed from a precursor compound and/or is a structural or chemical analog. All states of matter, as used herein, such as solid, liquid and gas relate to the specified state of matter at 25° C. and atmospheric pressure.

The term “skin” as used herein includes the skin on the face, neck, chest, back, arms, axilla, buttocks, hands, legs and scalp. Skin benefit agent, as used herein, is meant to include a component that improves a facial or body characteristic after topical application like a skin characteristic and/or benefits the same wherein the skin benefit agent can be incorporated in a desirable format.

The following Examples are provided to further illustrate an understanding of the invention. The Examples are not intended to limit the scope of the claims. One of ordinary skill in the art will recognize that variations of the method steps that differ from the examples given may be practiced without deviating from the teachings of the present compositions.

### EXAMPLES

All combinations were made by mixing with moderate shear the mentioned active ingredients under conditions of about 25° C., and atmospheric pressure.

### Example 1: Combinations of Actives

| | Laminaria | Lindera | Olea |
|---|---|---|---|
| | Digitata | strychnifolia | europaea |
| Treatments | extract | extract | extract |
| | Weight percent (wt%) | | |
| Combo 1 | 2.00 | 3.00 | 1.25 |
| Combo 2 | 1.00 | 1.50 | 0.63 |
| Combo 3 | 0.05 | 0.08 | 0.03 |
| Combo 4 | 0.67 | 1.00 | 0.42 |
| Combo 5 | - | - | 1.25 |
| Combo 6 | - | 1.00 | - |
| Combo 7 (no actives) | - | - | - |

In this particular example, the Laminaria digitata extract employed is made commercially available by Chemyunion under the tradename Energilium 3R, the Lindera strychnifolia (root) extract employed is made commercially available by SILAB under the tradename Circagenyl®, the Olea europaea extract employed is made commercially available by Barnet Products LLC under the tradename Olivys-EA.

### Example 2: Procollagen Levels in Human Dermal Fibroblasts

The effect of the method for treating skin disclosed herein to stimulate procollagen production was evaluated using media cultures of human dermal fibroblasts in 96-well plates. Each well was dosed with the candidate active combinations (Combos 1 through 7). Combination 7 is a control treatment, comprising none of the disclosed actives. If LED light radiation was to be applied, on the same day as treatment with the assigned active combinations, relevant wells were treated with a source of LED light radiation obtained from commercially available devices such as those from Celluma® LED Device on wrinkle mode at a pulsed setting at a set distance close to the plate. The main emission in ‘wrinkle mode’ is red light having a wavelength of 640 nm. Wrinkle mode also includes additional emissions of light at different wavelengths and lower amounts, such as blue light (465 nm) and near-infrared light (880 nm). On the next (second) day, LED exposure was repeated for each well. On the third day, an assay for procollagen was performed. PIP refers to procollagen type-I carboxy-terminal peptide and the assay measures the procollagen-I levels in nanograms/milliliter (ng/mL).

TABLE I
Procollagen-I levels in fibroblasts treated with combinations and no LED

| | Average PIP | Standard | Student's t-test | |
|---|---|---|---|---|
| Treatments | (ng/mL) | Deviation | Treatments compared | p-value |
| Combo 1 | 176.2 | 10.1 | Combo 7 vs Combo 1 | 0.251 |
| Combo 2 | 185.0 | 31.8 | Combo 7 vs Combo 2 | 0.247 |
| Combo 3 | 143.1 | 21.8 | Combo 7 vs Combo 3 | 0.276 |
| Combo 4 | 136.5 | 17.9 | Combo 7 vs Combo 4 | 0.198 |
| Combo 5 | 124.3 | 23.2 | Combo 7 vs Combo 5 | 0.121 |
| Combo 6 | 168.6 | 14.9 | Combo 7 vs Combo 6 | 0.388 |
| Combo 7 | 159.5 | 37.8 | - | - |

TABLE II
Procollagen-I levels in fibroblasts treated with combinations
and LED exposure for 30 minutes

| | | | Student's t-test | |
|---|---|---|---|---|
| | Average PIP | Standard | Treatments | p- |
| Treatments | (ng/mL) | Deviation | compared | value |
| Combo 1 | 241.0 | 33.8 | Combo 7 (with no LED | 0.251 |
| | | | exposure) vs Combo 1 | |
| Combo 2 | 152.4 | 11.8 | Combo 7 (with no LED | 0.247 |
| | | | exposure) vs Combo 2 | |
| Combo 3 | 227.0 | 20.7 | Combo 7 (with no LED | 0.276 |
| | | | exposure) vs Combo 3 | |
| Combo 4 | 154.8 | 31.6 | Combo 7 (with no LED | 0.198 |
| | | | exposure) vs Combo 4 | |
| Combo 5 | 145.7 | 16.9 | Combo 7 (with no LED 5 | 0.121 |
| | | | exposure) vs Combo | |
| Combo 6 | 172.2 | 17.3 | Combo 7 (with no LED | 0.388 |
| | | | exposure) vs Combo 6 | |
| Combo 7 | 166.0 | 25.6 | Combo 7 (with no LED | 0.407 |
| | | | exposure) vs Combo 7 | |
| Combo 7 | 159.5 | 37.8 | - | - |
| (with no | | | | |
| LED | | | | |
| exposure) | | | | |

The data in Example 2 unexpectedly demonstrates that in each case, when combined with LED exposure for 30 minutes, Combinations 1 and 3 in particular resulted in superior PIP boosting in human dermal fibroblasts when compared to controls (both in presence and absence of LED exposure for 30 minutes). The increase in average PIP levels, that is increased procollagen production in the skin, results in smoother and more elastic skin.

1. A method for increasing procollagen production comprising:
(a) topically applying to a surface of skin in need of treatment a cosmetic composition comprising (i) from 0.001 to 10%, by weight of seaweed-derived active, (ii) from 0.001 to 10%, by weight of a first botanical active, (iii) from 0.001 to 10%, of a second botanical active, and (iv) a cosmetically acceptable vehicle; and
(b) applying to the skin a source of light radiation originating from a light-emitting diode (LED) having wavelength ranging from 300 nm to 800 nm, for 10 to 60 minutes.

2. The method according to claim 1, wherein the seaweed-derived active comprises Laminaria cloustoni, Laminaria digitata, Laminaria hyperborea, Laminaria japonica, Laminaria ochroleuca, Laminaria saccharina, or mixtures thereof.

3. The method according to claim 1, wherein the seaweed-derived active further comprises at least one of magnesium aspartate, disodium adenosine triphosphate, or a combination thereof.

4. The method according to claim 1, wherein the first botanical active comprises Lindera aggregate, Lindera latifolia, Lindera obtusiloba, Lindera strychnifolia, or mixtures thereof.

5. The method according to claim 1, wherein the first botanical active comprises Lindera strychnifolia (root) extract.

6. The method according to claim 1, wherein the second botanical active comprises olive extract.

7. The method according to claim 1, wherein a weight ratio of the seaweed-derived active to the first botanical active is from 1:3 to 4:5, and wherein a weight ratio of the seaweed-derived active to the second botanical active is from 5:2 to 1:1.

8. The method according to claim 1, wherein the light radiation is LED red light.

9. The method according to claim 1, wherein the method comprises an optional step of wiping or rinsing off of the cosmetic composition topically applied to the skin after exposure to light radiation.

10. The method according to claim 1, wherein the cosmetically acceptable vehicle is aqueous or an emulsion.

11. The method according to claim 1, wherein the cosmetic composition is in the form of a cream, a lotion, or a gel.

12. (canceled)

13. A cosmetic treatment kit for increasing procollagen production, wherein the kit comprises:
(a) a cosmetic composition comprising (i) 0.001 to 10%, by weight of seaweed-derived active, (ii) from 0.001 to 10%, by weight of a first botanical active, (iii) from 0.001 to 10%, of a second botanical active, and (iv) a cosmetically acceptable vehicle, and
(b) a device for cosmetic treatment by light comprising at least one source of light-emitting diode (LED) light radiation at a wavelength of 300 nm to 800 nm.

14. (canceled)

15. (canceled)

16. The method according to claim 7, wherein the weight ratio of the seaweed-derived active to the first botanical active is from 1:2 to 3:4.

17. The method according to claim 7, wherein the weight ratio of the seaweed-derived active to the second botanical active is from 2.1 to 5:4.

18. A method for increasing procollagen production comprising:
(a) topically applying to a surface of skin in need of treatment a cosmetic composition comprising (i) from 0.04 to 2.5%, by weight of seaweed-derived active, wherein the seaweed derived active is Laminaria digitata, (ii) from 0.04 to 3.5%, by weight of a first botanical active, wherein the first botanical active is Lindera strychnifolia, (iii) from 0.03 to 2%, of a second botanical active, wherein the second botanical extract is Olea europaea extract, and (iv) a cosmetically acceptable vehicle; and
(b) applying to the skin a source of light radiation originating from a light-emitting diode (LED) having wavelength ranging from 300 nm to 800 nm, for 10 to 60 minutes.

19. The method according to claim 18, wherein a weight ratio of the seaweed-derived active to the first botanical active is from 1:3 to 4:5.

20. The method according to claim 19, wherein the weight ratio of the seaweed-derived active to the first botanical active is from 1:2 to 3:4.

21. The method according to claim 18, and wherein a weight ratio of the seaweed-derived active to the second botanical active is from 5:2 to 1:1.

22. The method according to claim 21, and wherein the weight ratio of the seaweed-derived active to the second botanical active is from 2.1 to 5:4.
